(19) United States
(12) Patent Application Publication
(10) Pub. No.: US 2004/0014733 A1
(43) Pub. Date: Jan. 22, 2004

(54) Hydrothermically processed compositions containing phytosterols

(76) Inventors: Thomas P. Binder, Decatur, IL (US); Thomas V. Gottemoller, Mt. Zion, IL (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/410,193

(22) Filed: Apr. 10, 2003

Related U.S. Application Data
(60) Provisional application No. 60/371,161, filed on Apr. 10, 2002.

(51) Int. Cl. A61K 31/56

(52) U.S. Cl. 514/169

(57) ABSTRACT

Hydrothermically formed phytosterol-emulsifier compositions are disclosed, along with the process for their production. The compositions are organoleptically pleasing and useful in foods, health products, and nutraceutical products for lowering cholesterol levels. Phytosterols are mixed with an emulsifier dispersion and then hydrothermically heated to integrate the phytosterols into a micellar form with the emulsifier, which produces a smooth and pleasing mouthfeel and a bioactive and bioavailable product.

### CROSS-REFERENCE TO RELATED APPLICATIONS

This application claims the benefit of U.S. Provisional Application No. 60/371,161 filed Apr. 10, 2002, the content of which is incorporated herein by reference.

### BACKGROUND OF THE INVENTION

1. Field of the Invention

The present invention relates to compositions for improved health and decreased cholesterol absorption, the compositions comprising hydrothermically processed phytosterols. More particularly, the compositions of the invention comprise phytosterols in a dispersion of emulsifiers, the combination of which is processed through a hydrothermic heating process.

2. Related Art

Phytosterols are plant sterols structurally similar to cholesterol that have been known for many years to reduce cholesterol absorption and serum cholesterol levels while not being absorbed themselves. Lowering of circulating cholesterol and low density lipoprotein cholesterol is an important part of a strategy to prevent and treat cardiovascular disease and especially coronary heart disease. Cholesterol absorption is a critical component of whole body cholesterol metabolism. Cholesterol derived from the diet and also from endogenous biliary secretion enters the intestine, and approximately 50% of the mixed intestinal load is absorbed. Bosner, M. S., Ostlund, R. E., Jr., Osofisan, O., Grosklos, J., Fritschle, C., Lange, L. G. 1993. The failure to absorb cholesterol quantitatively is therefore a key mechanism for the elimination of cholesterol from the body.

Drugs commonly used to treat high cholesterol levels have little or no effect on cholesterol absorption. For example, the potent new hydroxymethylglutaryl coenzyme A reductase inhibitors have a primary action to reduce cholesterol synthesis rather than increase cholesterol elimination. Bile acid sequestrants such as the ion-exchange resin cholestyramine act within the intestine but do not bind cholesterol and may actually increase cholesterol absorption when given chronically. McNamara, D. J., et al., J. Lipid Res. 21:1058-1064 (1980). Although orally-administered neomycin reduces cholesterol absorption effectively, it is toxic and has the disadvantage of requiring chronic administration of a potent antibiotic. Samuel, P., N. Engl. J. Med. 301:595-597 (1979). The drug Cytellin.RTM., an aqueous suspension of mixed phytosterols, was produced by Eli Lilly Co. for treatment of elevated cholesterol, but it has not been sold since 1985. As seen, it is apparent that new inhibitors of cholesterol absorption would complement the currently available treatment for high serum cholesterol.

Since phytosterols are natural products which are non-toxic and byproducts of food processing, they may be important in the treatment of individuals with mildly-increased serum cholesterol, or for the general population in food products or dietary supplements. The use of phytosterols could reduce the need for drugs absorbed systemically.

Despite their potential attractiveness, the usefulness of phytosterols has been limited by small and erratic effectiveness and a large dosage requirement. For example, doses of 5-18 g sitosterol/day reduced serum cholesterol by 16-20%. Farquhar, J. W. and M. Sokolow, 1958. A dose-response study showed that 3-9 g/day of powdered sitosterol was needed to decrease serum cholesterol levels by 12%. Lees, A. M., et al., Atherosclerosis 28:325-338 (1977). To reduce the amount needed, recent experiments have used sitostanol instead of sitosterol because it appears to be more potent than other phytosterols and is non-absorbable. Sugano, J., et al., J. Nutr. 107:2011-2019 (1977). In subjects with severe hypercholesterolemia, sitostanol at 1.5 g/day reduced serum cholesterol by 15%. Heinemann, T., et al., Atherosclerosis 61:219-223 (1986). However, sitostanol at 3 g/day had no effect in subjects with moderate hypercholesterolemia. Denke, M. A., Am. J Clin. Nutr. 61:392-396 (1995).

Several investigators have proposed ways to increase the solubility or bioavailability of phytosterols in order to make them more useful. Based on studies in rats and the finding that phytosterol esters are much more soluble in oil than the free sterols, it has been proposed to use phytosterol esters in oil to lower cholesterol absorption. Mattson, F. H., et al., J. Nutr. 107:1139-1146 (1977). U.S. Pat. No. 5,502,045 describes the use of sitostanol ester in oil for the treatment of hypercholesterolemia in humans. Also, it was found that 2.8 g sitostanol/day given as sitostanol ester in margarine reduced LDL cholesterol by 16%. Miettinen, T. A., et al., N. England J. Med. 333:1308-1312 (1995). However, the use of sitostanol ester dissolved in dietary fat has the disadvantage of requiring the administration of 2350 g/day of dietary fat and of being 21% less effective at reducing cholesterol absorption in humans compared to the unesterified sterol. Mattson, F. H., et al, Am. J. Clin. Nutr. 35:697-700 (1982).

Other workers have investigated ways to improve the usefulness of unesterified phytosterols. In WO 95/00158, a complex of sitosterol and the unabsorbable dietary fiber pectin reduced serum cholesterol by 16.4% when given to hypercholesterolemic humans in a dose of 2.1 g/day. However, no measurements of an effect on cholesterol absorption were made, and the complex was only about 50% soluble even at strongly alkaline pH, suggesting that the bioavailability of the sitosterol component was limited.

U.S. Pat. No. 5,244,887 describes the use of stanols including sitostanol in food additives to reduce cholesterol absorption. In the '887 patent, for preparation of the additives, sitostanol is dissolved with an edible solubilizing agent such as triglyceride, an antioxidant such as tocopherol, and a dispersant such as lecithin, polysorbate 80, or sodium lauryl sulfate. However, no data were given to guide one in the selection of the most effective components and their amounts or specific methods of preparation. Effectiveness in reducing cholesterol absorption was also not determined. The preferred embodiment consisted of 25% by weight stanols in vegetable oil, but the solubility of sterols in oil is only 2%.

U.S. Pat. No. 6,129,944 relates to a method for producing an edible product containing phytosterols and a carbohydrate sweetener. The present invention differs from the '944 patent, for example, in that the phytosterol composition of the present invention contains an emulsifer. Another advantage of the present invention is that it optimizes the processing and function of the phytosterol-emulsifier composition through hydrothermic processing.

U.S. Pat. No. 5,118,671 describes the production of sitosterol-lecithin complexes for pharmaceutical use but does not consider oral use for cholesterol lowering.

Cholesterol is absorbed from an intestinal micellar phase containing bile salts and phospholipids which is in equilibrium with an oil phase inside the intestine. Esterification of the phytosterol with delivery through the oil phase of foods is one method of providing for the delivery of phytosterols, but it has the disadvantage of use of edible oils as the carrier.

U.S. Pat. Nos. 5,932,562 and 6,063,776 provide a delivery system for plant sterols, particularly sitostanol, which avoids an oil phase and which provides bioavailable sitostanol at a level which reduces cholesterol absorption as much as 37%. The '767 patent also discloses that an emulsifier with certain taste characteristics is used in as low amounts as possible.

The '562 patent and the '776 patent further provide a water soluble composition which provides sitostanol, not dissolved in fat, but rather combined with an emulsifier, such as a lecithin and lysolecithin mix (the '562 patent) or sodium stearoyl 2-lactylate (“SSL”) (the '776 patent), in an aqueous vesicular complex that can enter directly into the intestinal micellar phase and is therefore highly bioavailable.

The '562 and '776 patents also provide a composition of enhanced solubility that contains a plant sterol, such as sitostanol, mixed with an emulsifier such as phospholipids (the '562 patent) or an emulsifier other than a phospholipid, namely SSL (the '776 patent), which has water solubility in excess of 90%.

The '562 and '776 patents also provide a method for reducing cholesterol absorption from food products containing cholesterol by mixing finely divided water soluble powder of an aqueous homogeneous micellar mix of sitostanol and lecithin (the '562 patent) or SSL (the '776 patent) with a food product.

The '562 and '776 patents also provide a method of manufacturing a dry, finely divided water soluble powder which contains a plant sterol, such as sitostanol, and lecithin, which is highly water soluble, so that when in contact with an aqueous system it will provide an aqueous vesicular complex which can enter directly into the intestinal micellar phase to inhibit cholesterol absorption.

It is an objective of the present invention to provide improved processing and other characteristics to the composition of the '562 and '776 patents through hydrothermic processing one or more phytosterols in a dispersion of one or more emulsifiers.

The method and manner of achieving each of the above objectives, as well as others, will become apparent from the detailed description of the invention that follows hereinafter.

### SUMMARY OF THE INVENTION

The present invention provides a process for making an organoleptically pleasing composition comprising phytosterols in a form that should retain bioactivity and bioavailability. The process involves the addition of phytosterols to a dispersion of one or more emulsifiers, and the subsequent hydrothermic processing of this phytosterol-emulsifier mixture. The resultant material contains the phytosterols in micellar form, and possesses a smooth and pleasing mouthfeel. Such a mouthfeel is not produced by the typical commercially available phytosterol-containing products unless the phytosterols have been chemically modified, for example by esterification.

The process of the present invention involves the production of an aqueous emulsifier dispersion. The emulsifier dispersion is blended with phytosterols, and in preferred embodiments the phytosterols have been spray prilled. The mixture is then subjected to hydrothermic processing. For purposes of the present disclosure, hydrothermic processing is intended to be a generic description of a process involving the heating, with steam, in an aqueous system and under pressure, of the phytosterol-emulsifier mixture, to a temperature of above 100° C. An alternative phrase that could be used to describe such a process would be jet cooking. For example, U.S. Pat. No. 5,936,069 discusses “jet cooking” processes. In one embodiment of the present invention, hydrothermic processing (“jet cooking”) can be carried out in an apparatus such as an APV Lab Media Sterilizer. The aqueous composition from the hydrothermic processing may then be used as is, in food, health, supplement, and nutraceutical products, or it can be used as a food product ingredient. Alternatively, this aqueous product may be dried, with or without added drying aids, to obtain a free-flowing, dispersible product.

### DETAILED DESCRIPTION OF THE INVENTION

The present invention relates to a process for producing a highly palatable phytosterol-emulsifier dispersion that can be used in food, health, supplement, and nutraceutical products. The process involves blending an emulsifier dispersion with phytosterols and hydrothermically processing this blend.

In an embodiment of the present invention, an emulsifier is mixed with water to form an aqueous emulsifier mixture. Any number of types of emulsifiers, preferably food grade emulsifiers, may be used in the practice of the present invention. In a preferred embodiment, the emulsifier is a low hydrophilic-lipophilic balance emulsifier. Emulsifiers such as lecithin, distilled monoglycerides, polyglycerol esters, propylene glycol esters, ethoxylated monoglycerides, sucrose esters, and like emulsifiers can be used in accordance with the present invention. Deoiled lecithin, monoglycerides, and diglycerides are particularly preferred emulsifiers.

The aqueous emulsifier solution is then mixed with phytosterols. In another embodiment, the emulsifier and phytosterols are combined with water at the same time. The term “phytosterol” is used herein in a broad sense to mean plant-derived sterol or stanol compounds, including certain derivatives thereof. Phytosterols include, for example, stigmasterol, spinasterol, campesterol, and the α, β, and γ forms of sitosterol. The ester forms of these compounds are also appropriate for use in the practice of the present invention. A stanol compound, such as sitostanol, for example, is also useful as the phytosterol component in the process of the present invention. The ester forms of phytostanols can also be used in accordance with the present invention.

In a preferred embodiment of the present invention, the ratio of aqueous emulsifier to phytosterol is in the range of about 0.2:1 to about 10:1 and more preferably from about 1:1 to about 5:1. In another preferred embodiment of the present invention, and particularly where higher relative levels of phytosterols are to be mixed with the aqueous emulsifier solution, the phytosterols are either ground to a powder or prilled before they are added to the aqueous emulsifier. The grinding or prilling improves incorporation of the phytosterols into the aqueous system at higher relative phytosterol levels. In another preferred embodiment, the phytosterols are prilled by atomizing molten phytosterols in a cool air stream. As those of skill in the art will recognize, the grinding or prilling decreases the particle size of the phytosterols, thereby improving incorporation into the aqueous system.

In one embodiment of the invention, the mixture of phytosterols in the aqueous emulsifier solution is then subjected to heat. It is preferred to heat the mixture to a temperature of about 40° C. to about 100° C.; it is even more preferred to heat the mixture to a temperature of about 80° C. to about 100° C.

Next, the mixture of phytosterols is subjected to hydrothermic processing (which may also be referred to as “jet cooking”). For the purposes of the present disclosure, “hydrothermic processing” is a generic description of a process that involves heating the phytosterol mixture, with steam, in an aqueous system and under pressure, to a temperature of above 100° C. Thus, the process involves high temperature processing, or jet cooking, in, for example, an APV lab media sterilizer. In a preferred embodiment, the temperature is from about 100° C. to about 200° C., and more preferably from about 135° C. to about 160° C. The hydrothermic processing times are typically short; preferred times range from about 2 seconds to about 10 minutes, and even more preferred times range from about 30 seconds to about 3 minutes. In a particularly preferred embodiment, a phytosterol-deoiled lecithin-maltodextrin composition was hydrothermically processed for 1.5 minutes at 152° C. As those of skill in the art will recognize, hydrothermic processing times and temperatures will vary depending upon the specific formulation being processed, and processing time may increase or decrease as processing temperature decreases or increases. The optimization of the hydrothermic processing parameters is well within the skill of the art in view of the teaching of the present disclosure.

In yet another embodiment, the hydrothermic processing is stopped by cooling the phytosterol-emulsifier composition to a temperature from about 80° C. to about 150° C. In a preferred embodiment, this cooling is accomplished using a flash cooler.

Following hydrothermic processing, the hydrothermically formed phytosterol-emulsifier composition of the present invention is preferably homogenized at least once, and more preferably twice. Either a single stage or a two stage homogenizer can be used, and preferred ranges of pressure for homogenization are from about 1,000 psi to about 10,000 psi. More preferably, the ranges are from about 2,000 psi to about 8,000 psi.

The hydrothermically formed phytosterol-emulsifier compositions of the present invention have a smooth, pleasant mouthfeel without graininess. The phytosterol-emulsifier compositions can be used in accordance with the present invention in an aqueous form for the preparation of food, health, supplement, and nutraceutical products that are produced using liquid ingredients. The present invention embodies health drinks and other beverages, frozen or fresh desserts, baked goods, meat products, and a variety of other products for consumption that could be formulated to include the hydrothermically formed aqueous phytosterol-emulsifier compositions.

In another embodiment of the invention, the hydrothermically formed aqueous phytosterol-emulsifier composition can be dried to form a dry, solid, or powdered form. The aqueous phytosterol-emulsifier composition can be dried using spray drying, flash drying, freeze drying, or any other art that is recognized as a drying method that would result in the production of a powder either directly, or indirectly through a subsequent grinding drying step. Drying aids, such as proteins or carbohydrates, including maltodextrin, can be added to the phytosterol composition in an embodiment of the present invention. The invention embodies adding drying aids before or after hydrothermic processing. Those of skill in the art would be familiar with the use of such drying aids. The amounts added can vary and, given the present disclosure, require merely the optimization of process parameters.

An embodiment of the present invention includes using the phytosterol powder as a functional food itself or using it as an ingredient in foods that can include dry powder as an ingredient. The powder form can also be reconstituted into an aqueous composition, which, upon reconstitution, again demonstrates a smooth mouthfeel, without graininess. The powder form possesses excellent shelf-life and stability, and therefore can be a preferred form for storage, bulk handling, shipping, and similar needs.

Having generally described the invention in the foregoing, the following examples are provided to further illustrate the invention in certain more specific embodiments. However, the examples are not intended to limit the full scope of the invention as claimed unless clearly stated otherwise.

### EXAMPLES

### Example 1

| | Pounds |
|---|---|
| Deoiled Lecithin | 3.33 |
| Sterols | 1.67 |
| 10 DE Maltodextrin | 5.00 |
| Water | 40.00 |
| | 50.00 |

1. Add Deoiled Lecithin to 49° C. water under good agitation.

2. Add Sterols and 10 DE Maltodextrin and agitate.

3. Heat mixture to 74° C.

4. Jet cook at 152° C. for 1.5 minutes and cool to 79° C. using a flash cooler.

5. Homogenize at 3500/500 psi (first/second stage).

6. Spray dry—T inlet=250° C. & T outlet=82° C.

### Example 2

| | Pounds |
|---|---|
| Deoiled Lecithin | 3.33 |
| Sterols | 1.67 |
| Water | 95 |
| | 100.00 |

The processing parameters for Example 2 were the same as those for Example 1.

### Example 3

| | Pounds |
|---|---|
| Mono-and diglycerides | 3.33 |
| Sterols | 1.67 |
| Water | 95 |

The processing parameters for Example 3 were the same as those for Example 1.

Having now fully described the present invention in some detail by way of illustration and example for purposes of clarity of understanding, it will be obvious to one of ordinary skill in the art that the same can be performed by modifying or changing the invention with a wide and equivalent range of conditions, formulations, and other parameters thereof, and that such modifications or changes are intended to be encompassed within the scope of the appended claims.

All publications, patents, and patent applications mentioned in this specification are indicative of the level of skill of those skilled in the art to which this invention pertains, and are herein incorporated by reference to the same extent as if each individual publication, patent, or patent application was specifically and individually indicated to be incorporated by reference.

What is claimed is:

1. A process for producing an aqueous phytosterol-emulsifier composition comprising:
(a) combining one or more phytosterols, one or more emulsifiers, and water to form a mixture; and
(b) hydrothermically processing said mixture to produce an aqueous phytosterol-emulsifier composition.

2. The process of claim 1, wherein said phytosterols (a) are selected from the group consisting of alpha, beta, and gamma forms of sitosterol, sitostanol, stigmasterol, stigmastanol, campesterol, campestanol, spinasterol, phytosterol esters, phytostanol esters, and mixtures thereof.

3. The process of claim 1, wherein said mixture (a) is heated prior to said hydrothermic processing (b).

4. The process of claim 3, wherein said heating occurs at a temperature from about 40° C. to about 100° C.

5. The process of claim 4, wherein said heating occurs at a temperature from about 80° C. to about 100° C.

6. The process of claim 1, wherein said hydrothermic processing (b) occurs at a temperature from about 100° C. to about 200° C.

7. The process of claim 6, wherein said hydrothermic processing (b) occurs at a temperature from about 135° C. to about 160° C.

8. The process of claim 7, wherein said hydrothermic processing (b) occurs at a temperature of about 150° C.

9. The process of claim 8, wherein said hydrothermic processing occurs for about 1 to about 2 minutes.

10. The process of claim 1, wherein said emulsifier (a) is mixed with said water (a) to obtain an aqueous emulsifier solution prior to said mixing (a) of said emulsifier and said water with said phytosterols (a).

11. The process of claim 1, wherein said emulsifier (a) is a low hydrophilic-lipophilic balance emulsifier.

12. The process of claim 11, wherein said emulsifier is selected from the group consisting of lecithin, modified lecithin, monoglycerides, diglycerides, distilled monoglycerides, polyglycerol esters, propylene glycol esters, ethoxylated monoglycerides, sucrose esters, and mixtures thereof.

13. The process of claim 12, wherein said emulsifier (a) is lecithin.

14. The process of claim 13, wherein said lecithin is deoiled lecithin.

15. The process of claim 1, wherein the ratio of said emulsifier (a) to said phytosterols (a) is from about 0.2:1 to about 10:1.

16. The process of claim 15, wherein the ratio of said emulsifier (a) to said phytosterols (a) is from about 1:1 to about 5:1.

17. The process of claim 16, wherein the ratio of said emulsifier (a) to said phytosterols (a) is about 2:1.

18. The process of claim 1, wherein said aqueous phytosterol-emulsifier composition (b) is cooled to a temperature from about 80° C. to about 150° C.

19. The process of claim 18, wherein said aqueous phytosterol-emulsifier composition (b) is cooled to a temperature of about 80° C.

20. The process of claim 1, wherein said aqueous phytosterol-emulsifier composition (b) is cooled by flash cooling.

21. The process of claim 1, wherein said aqueous phytosterol-emulsifier composition (b) is homogenized.

22. The process of claim 20, wherein said aqueous phytosterol-emulsifier composition (b) is homogenized after flash cooling said aqueous phytosterol-emulsifier composition.

23. The process of claim 21, wherein said homogenization is a single stage homogenization.

24. The process of claim 21, wherein said homogenization is a two stage homogenization.

25. The process of claim 21, wherein said homogenization occurs at a pressure from about 1,000 psi to about 10,000 psi.

26. The process of claim 25, wherein said homogenization occurs at a pressure from about 2,000 psi to about 8,000 psi.

27. The process of claim 1, wherein said aqueous phytosterol-emulsifier composition (b) is dried.

28. The process of claim 27, wherein said aqueous phytosterol-emulsifier composition (b) is dried to obtain a phytosterol powder composition.

29. The process of claim 27, wherein said aqueous phytosterol-emulsifier composition (b) is dried by spray drying, flash drying, or freeze drying.

30. The process of claim 27, wherein said aqueous phytosterol-emulsifier composition (b) comprises a drying aid.

31. The process of claim 30, wherein said drying aid comprises a protein.

32. The process of claim 30, wherein said drying aid comprises a carbohydrate.

33. The process of claim 1, wherein said aqueous phytosterol-emulsifier composition (b) comprises maltodextrin.

34. The process of claim 32, wherein said aqueous phytosterol-emulsifier composition (b) comprises maltodextrin.

35. The process of claim 1, wherein said phytosterols (a) are prilled prior to said combining (a) with said emulsifiers and said water.

36. The process of claim 1, wherein said phytosterols (a) are ground prior to said combining (a) with said emulsifiers and said water.

37. An edible product produced by the process of claim 1.

38. The product of claim 37, wherein said product is a beverage or pourable liquid.

39. The product of claim 37, wherein said product is a solid dry or semi-moist edible product.

40. An edible composition produced by the process of claim 1, wherein said composition reduces cholesterol absorption in animals or humans.

41. An edible composition produced by the process of claim 1, wherein said composition lowers serum cholesterol in animals or humans.

42. A process for producing a phytosterol composition comprising:
(a) combining one or more phytosterols, one or more emulsifiers, maltodextrin, and water to form a mixture;
(b) hydrothermically processing said mixture to produce an aqueous phytosterol composition; and
(c) drying said aqueous phytosterol composition to produce a phytosterol composition.

43. The process of claim 42, wherein said aqueous phytosterol composition is homogenized.

44. The process of claim 42, wherein said emulsifier (a) is lecithin.

45. The process of claim 44, wherein said lecithin is deoiled lecithin.

46. The process of claim 42, wherein said emulsifier (a) comprises monoglycerides and diglycerides.

47. The process of claim 42, wherein the ratio of said emulsifier (a) to said phytosterol (a) is from about 0.2:1 to about 10:1.

48. The process of claim 47, wherein the ratio of said emulsifier (a) to said phytosterol (a) is from about 1:1 to about 5:1.

49. The process of claim 48, wherein the ratio of said emulsifier (a) to said phytosterol (a) is about 2:1.

50. An edible product produced by the process of claim 42.

51. The product of claim 50, wherein said product is a solid dry or semi-moist edible product.

52. An edible composition produced by the process of claim 42, wherein said composition reduces cholesterol absorption in animals or humans.

53. An edible composition produced by the process of claim 42, wherein said composition lowers serum cholesterol in animals or humans.

54. A process for producing a phytosterol composition comprising:
(a) combining one or more emulsifiers and water to form an aqueous emulsifier dispersion;
(b) combining one or more phytosterols and maltodextrin with said aqueous emulsifier dispersion to form a mixture;
(c) hydrothermically processing said mixture to produce an aqueous phytosterol composition;
(d) homogenizing said aqueous phytosterol composition; and
(e) drying said aqueous phytosterol composition;
wherein said emulsifier is selected from the group consisting of deoiled lecithin, monoglycerides, diglycerides, and mixtures thereof, and wherein the ratio of said emulsifier (a) to said phytosterol (b) is from about 0.2:1 to about 10:1.

55. The process of claim 54, wherein the ratio of said emulsifier (a) to said phytosterol (b) is from about 1:1 to about 5:1.

56. The process of claim 55, wherein the ratio of said emulsifier (a) to said phytosterol (b) is about 2:1.

57. An edible product produced by the process of claim 54.

58. The product of claim 57, wherein said product is a solid dry or semi-moist edible product.

59. An edible composition produced by the process of claim 54, wherein said composition reduces cholesterol absorption in animals or humans.

60. An edible composition produced by the process of claim 54, wherein said composition lowers serum cholesterol in animals or humans.
